(19) United States
(12) Patent Application Publication
(10) Pub. No.: US 2018/0030516 A1
(43) Pub. Date: Feb. 1, 2018

(54) Microbiome Diagnostics

(71) Applicant: Alere Inc., Waltham, MA (US)

(72) Inventors: Namal Nawana, Waltham, MA (US); Julius C. Fister, III, Waltham, MA (US)

(21) Appl. No.: 15/553,704

(22) PCT Filed: Feb. 26, 2016
(86) PCT No.: PCT/US16/19681

§ 371 Date: Aug. 25, 2017

Related U.S. Application Data
(60) Provisional application No. 62/139,415, filed on Mar. 27, 2015.
(60) Provisional application No. 62/121,936, filed on Feb. 27, 2015.

(51) Int. Cl. C12Q 1/68

(52) U.S. Cl. CPC C12Q 1/689; C12Q 1/6806; C12Q 2600/118; C12Q 2600/158; C12Q 2600/16

(57) ABSTRACT

A method for determining data related to a microbiome of a human includes isothermally amplifying polynucleotides of at least N different microorganisms present in a sample obtained from the human, wherein N>1, determining, based on the amplified polynucleotides, microbiome data comprising data indicative of at least one of a presence and an abundance of each of the N microorganisms, and determining, based on the microbiome data and prior data related to the N microorganisms, data related to a condition of the animal.

### CROSS-REFERENCE TO RELATED APPLICATION

This application claims the benefit of U.S. Provisional Patent Application Ser. No. 62/121,936, filed on Feb. 27, 2015, and U.S. Provisional Patent Application Ser. No. 62/139,415, filed on Mar. 27, 2015, the entire contents of which are both hereby incorporated by reference.

### FIELD OF THE INVENTION

The invention relates to the microbiome of animals such as humans.

### BACKGROUND OF THE INVENTION

The Human Microbiome Project was established in 2008 with the aim of enabling the comprehensive characterisation of the human microbiome, described as the population of microbial species that inhabit the human being, both on the skin surface and within and throughout the body. Microbial communities have been characterised within the nasal passages, oral cavity, skin, gastrointestinal tract and urogenital tract. The composition of the microbiome of an individual, e.g., the number, identities, and relative abundance of members of the individual's microbiome, is correlated with conditions, e.g., health conditions, of the individual. There is a need for rapid and accurate determination of microbiome data of a given individual so that the resulting data can be used in a timely, meaningful way to benefit the wellbeing of the individual. For example, there is a need for determinations that can be made quickly, e.g., in 45 minutes or less, at the point of care, e.g., remote from a central laboratory, and using instruments that can be operated by individuals without substantial training.

### SUMMARY OF THE INVENTION

In some aspects, the invention relates to the microbiome of animals such as humans. In embodiments of the invention, a method includes amplifying polynucleotides of at least N different microorganisms present in a sample obtained from an animal and determining, based on the amplified polynucleotides, microbiome data. The microbiome data includes data indicative of at least one of a presence and an abundance of each of the N microorganisms. The method also includes determining, based on the microbiome data and on prior data related to the N microorganisms, data related to a condition of the animal.

In embodiments, the animal is a human being. In other embodiments, the animal is an animal of agricultural importance, e.g., a fowl, bovine, or porcine, or an animal of human companionship, e.g., a feline or canine.

The abundance of a microorganism may be an absolute abundance, e.g., a number per volume, or a relative abundance, e.g., an abundance of a microorganism relative to an abundance of one or more other microorganisms in the sample or other parameters. Data indicative of the presence of a microorganism may indicate the absence of such microorganism.

In embodiments, the amplification of the polynucleotides is performed isothermally. For example, the amplification may be performed without subjecting the polynucleotides to thermal cycling, e.g., by maintaining a temperature of the polynucleotides to within +/−20° C., to within +/−15° C., to within +/−10° C., to within +/−5° C., to within +/−2.5° C., or at a substantially constant temperature during amplification.

In embodiments, the amplification of the polynucleotides is performed without subjecting the polynucleotides to a temperature sufficient to denature double stranded polynucleotides during the amplification. For example, the amplification of the polynucleotides may be performed without subjecting the polynucleotides to a temperature in excess of about 90° C., about 80° C., about 70° C., or about 60° C. during amplification. In embodiments, the amplification of the polynucleotides is performed without subjecting the polynucleotides to conditions sufficient to denature double stranded polynucleotides during the amplification. For example, the amplification may be performed without subjecting the polynucleotides to physical, chemical, or thermal conditions sufficient to denature double stranded polynucleotides during amplification.

In embodiments, the amplification of the polynucleotides is performed without first subjecting the polynucleotides to a temperature sufficient to denature double stranded polynucleotides present in the sample. For example, the amplification of the polynucleotides may be performed without first subjecting the polynucleotides to a temperature in excess of about 90° C., about 80° C., about 70° C., about 60° C., or about 55° C. In embodiments, the polynucleotides and/or amplicons thereof are detected without first subjecting the polynucleotides to such excess temperatures. In embodiments, the amplification of the polynucleotides is performed without first subjecting the polynucleotides to conditions sufficient to denature double stranded polynucleotides present in the sample. For example, the amplification may be performed without first subjecting the polynucleotides to physical, chemical, or thermal conditions sufficient to denature double stranded polynucleotides present in the sample.

In embodiments, isothermal amplification is performed using recombinase polymerase amplification (RPA) or nicking and extension amplification reaction (NEAR).

In embodiments, the amplification is performed in a total time T beginning with a step of combining the polynucleotides with reagents sufficient to perform the amplification and ending when amplification has proceeded by an amount sufficient to permit the qualitative or quantitative determination of the polynucleotides or amplicons thereof. In other embodiments, the total time T begins with a step of combining microorganisms comprising the polynucleotides of at least some of the N microorganisms with reagents sufficient to release the polynucleotides from the microorganisms. For example, the microorganisms may be cells, e.g., bacterial, fungal, or yeast cells, and the reagents may be lysing reagents. In other embodiments, the total time T begins with the release of polynucleotides of at least some of the N microorganisms from the microorganisms, e.g., time T may begin with lysis of the microorganisms. In any of such embodiments, the total time T may be about 45 minutes or less, about 30 minutes or less, about 20 minutes or less, or about 15 minutes or less.

In embodiments, the amplification of the polynucleotides includes amplifying the polynucleotides by at least about 10⁶ fold, at least about 10⁷ fold, at least about 10⁸ fold, at least about 10⁹ fold, at least about 10¹⁰ fold, at least about 10¹¹ fold, or at least about 10¹² fold. Such amplification may be performed within the time T.

In embodiments, N, the number of different microorganisms, is at least 3, at least 5, at least 7, at least 10, at least 15, at least 20, or at least 25. In embodiments, at least N1 of the N microorganisms are different bacteria. N1 may be at least 3, at least 5, at least 7, at least 10, at least 15, at least 20. N1 may be at least 50%, at least 75% of N. N1 may equal N. In embodiments, a number N2 of the N microorganisms are different microorganisms other than bacteria. N2 may be at least 1, at least 3, at least 5, at least 7, at least 10, at least 15. N2 may be at least 25%, at least 50%, at least 75% of N. N2 may equal N. The N2 different microorganisms other than bacteria may include one or more viruses, fungi, or yeast.

In embodiments, amplification of each of at least a number N5 of the polynucleotides of the N microorganisms is performed in the same volume. N5 may be at least 3, at least 5, at least 7, at least 10, at least 15, at least 20. N5 may be at least 50%, at least 75% of N. N5 may equal N. The volume in which amplification is performed may be about 1 mL or less, about 750 μL or less, about 500 μL or less, about 250 μL or less, or about 100 μL or less.

In embodiments, the volume is a solution phase and the amplification performed in the solution phase is a first amplification performed using mobile, e.g., diffusible, reagents, which may include primers and/or probes A second amplification is performed adjacent a solid phase using immobilized reagents, which may include primers and/or probes immobilized to a surface.

In embodiments, the surface includes an array of reagents, e.g., primers and/or probes, immobilized thereto, e.g., unable to freely diffuse away from the surface, with each site of the array comprising reagents configured to amplify and/or detect polynucleotides related to one of the N microorganisms. In embodiments, the solution phase includes a number of particles, with the particles comprising immobilized reagents configured to amplify and/or detect polynucleotides related to a different one of the N microorganisms. For example, the reagents at sites of the array or of the particles may be configured to amplify amplicons produced by amplification in the solution phase and to permit detection thereof.

In embodiments, the method includes optically detecting amplicons at sites of the array using a two-dimensional optical detector configured to detect light emitted from sites of the array, e.g., fluorescence emitted by probes associated with the amplicons. In embodiments, the method includes electrically or electrochemically detecting amplicons at sites of the array by detecting an electrical property, e.g., a potential or current flow, associated with an electrode in electrical communication with each site of the array. In embodiments, the method includes optically detecting amplicons on particles, e.g., by sequentially illuminating the particles with light and detecting emitted fluorescence as in flow cytometry.

In embodiments, the volume in which the first, solution phase, amplification is performed is in fluidic communication during the first amplification with the solid phase at which the second, solid phase, amplification is performed. For example, the solid phase may be a wall of a chamber defining the volume in which the solution phase amplification occurs. The array may be disposed on the wall of the chamber. As another example, the solid phase may be particles suspended in the solution phase volume during the amplification.

In embodiments, the volume in which the first, solution phase, amplification is performed is in fluidically separated during the first amplification from the solid phase at which the second, solid phase, amplification is performed. For example, the solid phase may be a wall of a channel downstream from the volume in which the solution phase amplification occurs. The array may be disposed on the wall of the channel. Amplicons from the first amplification are brought, e.g., by fluid mass transport, from the volume, into fluidic contact with the surface.

In embodiments, the volume is a chamber of a microfluidic cartridge. The cartridge may include reagents sufficient to perform the amplification of the polynucleotides of the N5 microorganisms. The reagents may be in dry form within the cartridge prior to introduction of the polynucleotides to the cartridge.

In embodiments, the volume is a first solution phase and the amplification performed in the solution phase is a first amplification performed using mobile reagents, which may include primers and/or probes, and the second amplification is performed in a second solution phase in fluid communication with the first solution phase using mobile reagents, which may include primers and/or probes, within the second solution phase. For example, the first solution phase may include a first liquid solvent and the second solution phase may include a second liquid solvent with the first and second solvents being immiscible. The polynucleotides are soluble in each of the first and second solvents and, therefore, are able to diffuse across an interface there between. The second solution phase may include droplets of the second solvent suspended or otherwise distributed within the first solvent with the droplets comprising immobilized reagents configured to amplify and/or detect polynucleotides related to a different one of the N microorganisms. Amplicons present in the droplets of the second solvent may be detected optically as for particles of the solid phase.

In embodiments, the prior data includes data indicative of polynucleotides, e.g., genomes, of microorganisms isolated from samples obtained from one or more sites of each of multiple animals. The animals may be all of the same species. In embodiments, the animals include, e.g., may consist entirely of, human beings. In other embodiments, the animals include, e.g., may consist of fowl, bovine, porcine, feline, or canine animals.

In embodiments, the sites or samples of the animals from which microorganisms used to generate the prior data were obtained are the same site or sites of the animal from which the sample is obtained in respect of the condition. Typically, the animals from which microorganisms were obtained and used to generate the prior data are of the same species as the animal from which the polynucleotides are obtained in respect of the condition of the animal. For example, if the method is performed to determine data concerning the condition of a human, the method typically involves using prior data obtained from other humans.

In embodiments, the prior data includes data indicative of a correlation between the presence or abundance of the N microorganisms and the condition of the animal. For example, the data indicative of the condition of the animal may include data indicative of how the presence and abundance of such microorganisms in the sample obtained from the animal differ from a presence and abundance of the microorganisms found to be indicative of the presence, absence or severity of or a tendency toward the condition in the animals used to obtain the prior data.

In embodiments, the method further includes determining a treatment for the condition based at least in part on the microbiome data and the prior data. For example, the method may further include selecting and preferably administering a medicament to alter the presence or abundance of microorganisms in the animal depending on the presence, absence or severity of or a tendency toward the condition indicated by the prior data and the microbiome data. Alternatively or in combination, the method may further include modifying, e.g., breaking down or neutralizing, chemicals that are emitted by the microorganisms and which are connected to the condition of the animal. Exemplary medicaments include probiotics, pre-biotics, and antibiotics.

In embodiments, the prior data includes data indicative of at least a number N3 microorganisms obtained from each of at least a number N4 animals. In embodiments, N3 is at least 5, at least 10, at least 15, at least 20, at least 30, at least 50, at least 100, at least 500, at least 1000. In embodiments, N3 is at least as large as, e.g., equal to, N. In embodiments, the prior data includes data indicative of the genomes or other polynucleotides of the N3 microorganisms. The prior data includes 16S ribosomal RNA gene sequences of the microorganisms. In embodiments, N4 is at least 10, at least 50, at least 100, at least 500, at least 1000. The N4 animals are typically of the same species, e.g., homo sapiens, as the animal for which the method is performed.

In embodiments, the sample is obtained from one or more sites of the animal. In embodiments, the site is an oral cavity of the animal, e.g., one or more of the gingiva, buccal mucosa, hard palate, tonsils, saliva, subgingival plaque, supragingival plaque, throat, or tongue dorsum. In embodiments, the site is the nasal cavity, e.g., nostrils. In embodiments, the site is a portion of the skin of the animal, e.g., one or more of the scalp, the inner elbow, an interdigital space of the hand or foot, the ear canal, behind the ear, or hair, e.g., of the scalp, genitalia, e.g., vagina, or limbs. The site may be a nail of the animal, e.g., the hyponychium or lunula. In embodiments, the site is a portion of the urogenital tract of the animal, e.g., one or more of the urethra, the posterior fornix, the vaginal introitus. The site may be a digestive tract of the animal, e.g., one or more of the esophagus, stomach, small intestine, or large intestine. The site may be a joint of the animal, e.g., a bursa, a meniscus, or an articular capsule. The site may be a reproductive tract of the animal, e.g., the cervix, the vagina, the labium minus, or the labium majus. The sample may include urine, blood, stool, saliva, mucous, sputum, spinal fluid, tear fluid, a cheek swab, a throat swab, cerebrospinal fluid, or synovial fluid.

In embodiments, the sample is obtained from one or more sites of the animal known or suspected to be associated with a condition, e.g., a disease, of the animal. For example, the sample may be obtained from a site associated with a cancer or pre-cancerous condition, e.g., a skin lesion known or suspected of being associated with melanoma.

In embodiments, the determination of the data related to the condition of the animal is further based on factors or criteria in addition to and different from the microbiome data and the prior data related to the N microorganisms. For example, determination of the data about the condition of the animal may be further based on genetic background, location, habitat, age, gender, weight, known conditions, pigmentation, skin type, and hair type of the animal. Such data determined from other factors or criteria may be combined with the data based on the microbiome data and the prior data related to the N microorganisms in order to determine the data indicative of the condition of the animal.

In embodiments, the condition is an inflammatory skin disease, e.g., an inflammatory skin disease selected from the group consisting of acne, eczema, psoriasis, and sebaceous cysts. For example, the prior data may include data indicative of the presence or abundance of strains of N3 microorganisms, including from strains of propionibacterium acnes, in samples obtained from a number N4 animals and the microbiome data may include data indicative of the presence and abundance of at least a subset of the N3 microorganisms in the sample obtained from the animal. The data indicative of the condition of the animal may include data indicative of how the presence and abundance of such microorganisms in the animal differ from a presence and abundance of the microorganisms found to be indicative of the presence, absence or severity of or a tendency toward acne in the animals used to obtain the prior data. In embodiments, the method further includes determining a treatment for the inflammatory skin disease based at least in part on the microbiome data and the prior data. For example, the method may further include selecting and preferably administering a medicament to alter the presence or abundance of microorganisms in the animal.

In embodiments, the condition is dandruff. For example, the prior data may include data indicative of the presence or abundance of strains of N3 microorganisms, including from strains of Propionibacterium acnes and Staphylococcus epidermidis, while Malassezia restricta, in samples obtained from a number N4 animals and the microbiome data may include data indicative of the presence and abundance of at least a subset of the N3 microorganisms in the sample obtained from the animal.

In embodiments, the method includes providing a sample of hair, hair follicle or scalp swab, processing the sample to release polynucleotides present therein, analysing the sample using nucleic acid amplification to amplify the polynucleotides, and providing a result in 15 minutes or less identifying the presence or absence of at least one microorganisms, such as Malassezia globosa. The method may further include determining a treatment for the dandruff based at least in part on the microbiome data and the prior data. For example, the method may further include providing a cosmetic product, e.g., a hair cleansing product such as a shampoo, intended to inhibit metabolism of at least one microorganism, e.g., Malassezia globosa, thereby reducing its effect on the production or presence of dandruff.

In embodiments, the condition is a skin condition related to an appearance or moisture content of the skin, e.g., a dryness of the skin. For example, the prior data may include data indicative of the presence or abundance of strains of N3 microorganisms, including at least from some of strains of actinobacteria, firmicutes, proteobacteria, bacteroidetes, Propionibacteria, Staphylococci, Corynebacteria, b-Proteobacteria, and Flavobacteriales, in samples obtained from a number N4 animals and the microbiome data may include data indicative of the presence and abundance of at least a subset of the N3 microorganisms in the sample obtained from the animal.

In embodiments, the condition relates to an appearance, e.g., visual or olfactory, of the animal. For example, the condition may relate to an appearance of the skin, e.g., a moisture content of the skin or to an olfactory sensation determined, for example, by the presence of volatile organic compounds emanating from the skin. The method further includes selecting a product, e.g., a cosmetic product or dietary substance, based at least in part on the microbiome data and the prior data. For example, the cosmetic product may be selected to alter the presence or abundance of at least some microorganisms found to be indicative of a moisture content of skin including, for example, at least some of presence or abundance of strains of N3 microorganisms, including at least from some of strains of Propionibacteria, Staphylococci, Corynebacteria, b-Proteobacteria, and Flavobacteriales in samples obtained from a skin of a number N4 animals and the microbiome data may include data indicative of the presence and abundance of at least a subset of the N3 microorganisms in the sample obtained from the animal. As another example, the product may be selected to alter the presence or abundance of at least some microorganisms found to be indicative of the presence or absence of volatile organic compounds found unpleasant including at least some of Staphylococcus epidermidis and Bacillus subtilis. Alternatively or in combination, the product may be selected to alter, e.g., break down or neutralize, chemicals produced by the microorganisms including sulphur-containing organic compounds or organic acids including, for example, priopionic acid or isovaleric acid. In embodiments, the product is a cosmetic including at least one of a moisturizer, a cleansing agent, a deodorant, or an antiperspirant.

In embodiments, the method further includes determining a type or category of personal care product for the human. The method includes analyzing the microbiome data and the prior data to determine a correlation between (i) the identities, presence, and/or relative abundance of some or all of the N microorganisms and (ii) desirable properties of a personal care product for the human; and providing a recommendation for a personal care product to the human.

In embodiments, the condition is a dental condition, e.g., a dental condition selected from the group comprising caries, abscesses, gingivitis, and periodontitis. For example, the prior data may include data indicative of the presence or abundance of strains of N3 microorganisms, including at least from some of strains of Firmicutes, Bacteriodetes, Proteobacteria, Actinobacteria, Fusobacteria, Streptococcus, Abitrophia, Gemella, Granulicatella, Prophyromonas and P. gingivalis, in samples obtained from dental cavities of a number N4 animals and the microbiome data may include data indicative of the presence and abundance of at least a subset of the N3 microorganisms in the sample obtained from the animal. The method may further include determining a treatment for the dental condition based at least in part on the microbiome data and the prior data. For example, the treatment may including administering a medicament configured to modify the presence or abundance of one or more microorganisms found to be present (or absent) as compared to other microorganisms. Alternatively, or in addition, the medicament may be selected to alter, e.g., break down or neutralize, chemicals produced by the microorganisms present in the dental cavity. The medicament may be a dental care product such as a dental cleansing agent or an anti-bacterial agent.

In embodiments, the method includes screening a human subject to identify the presence of microorganisms known to be associated with prevalence of caries. The method may include providing a sample comprising oral fluid, dental plaque, cheek swab, the sample to release polynucleotides present therein, analysing the sample using nucleic acid amplification to amplify the polynucleotides, and providing a result in 15 minutes or less identifying the presence or absence of one or more of Actinomyces sp. strain B19SC, Streptococcus mutans, and Lactobacillus spp., Streptococcus parasanguinis, Abiotrophia defectiva, Streptococcus mitis, Streptococcus oralis, and Streptococcus sanguinis. If the presence of one or more of Actinomyces sp. strain B19SC, Streptococcus mutans, and Lactobacillus spp. is detected, the method may include providing a product that may inhibit the action of such species to cause or aggravate formation of caries or, if the presence of one or more of Streptococcus parasanguinis, Abiotrophia defectiva, Streptococcus mitis, Streptococcus oralis, and Streptococcus sanguinis is detected, the method may include offering a product that may enhance the growth of such species which are known to have beneficial effects on oral hygiene.

In embodiments, the condition is a condition of the digestive tract, e.g., a condition selected from the group consisting of inflammatory bowel disease, clostridium difficile infection, colorectal cancer, stomach or esophageal ulcers, or acid reflux. For example, the prior data may include data indicative of the presence or abundance of strains of N3 microorganisms, including at least from some of strains of H. pylori, in samples obtained from digestive tracts a number N4 animals and the microbiome data may include data indicative of the presence and abundance of at least a subset of the N3 microorganisms in the sample obtained from the animal.

In embodiments, the processor is configured to monitor a change in the microbial flora of the digestive tract in response to a dietary regimen designed to promote the proliferation of certain microbial species while inhibiting others. The processor may further be configured to provide real-time feed-back to a user that (i) the diet is having the desired effect; or (ii) whether intervention is required to alter dietary input in order to achieve the desired process of change to the microbiome. In cases where the microbiome is not evolving as desired or expected further intervention of a care professional may be required.

In embodiments, the processor is configured to aid in the determination of the likely glycaemic impact of particular food stuffs, when used in conjunction with suitable blood glucose or HbA1c test technology. Certain species present in the digestive tract may liberate glucose during transit of food through the gut, which may be adsorbed into the blood stream, potentially resulting adversely elevated blood glucose levels. The processor may be used to identify subjects at greatest risk, permitting appropriate action to minimise any long term effects of hyperglycaemia.

In embodiments, the processor may be used as an aid to controlling glycaemic status of an individual in conjunction with determination of HbA1c and/or blood glucose, particularly in cases where it has been demonstrated that an individual may be more sensitive to certain food groups or supplements than others, which as a consequence of the individual's digestive tract microbiome often results in adversely elevated blood glucose concentrations.

In embodiments, the processor is used as an aid to identifying whether an individual has an increased susceptibility to type-2 diabetes, when used in conjunction with either blood glucose and/or HbA1c test technology. Rapid determination of digestive track microbiome may permit diet and lifestyle management choices intended to mitigate hyperglycaemic episodes and maintain blood glucose within a desirable healthy range.

In embodiments, the condition is a metabolic condition, e.g., diabetes or obesity. For example, the prior data may include data indicative of the presence or abundance of strains of N3 microorganisms found to be correlated with the presence of pro-inflammatory cytokines, such as tumor necrosis factor-α (TNF-α), lipopolysaccharide activity, Toll-like receptor 4 (TLR4). The prior data may include data indicative of the presence or abundance of strains of N3 microorganisms including bacteroides-related bacteria, bifidobacteria, or firmicutes and the microbiome data may include data indicative of the presence and abundance of at least a subset of the N3 microorganisms in the sample obtained from the animal.

In embodiments, the condition is a condition of a joint of the animal, e.g., bursitis, arthritis, effusion, rheumatoid arthritis or a degenerative joint disease such as osteoarthritis.

In embodiments, the condition is a cardiac condition, e.g., a cardiovascular disease, including, atherosclerosis or atherosclerotic associated disease. In embodiments, the atherosclerotic associated disease is selected from the group consisting of coronary artery disease, myocardial infarction, angina pectoris, stroke, cerebral ischemia, intermittent claudication, gangrene, mesenteric ischemia, temporal arteritis, and renal artery stenosis.

In embodiments, the method includes screening a human subject to identify the presence, absence, and/or abundance of one or more microorganisms present in blood, tissue, plaque or other material obtained from the heart. The method may include providing a sample comprising blood, tissue, plaque or other material obtained from the heart, the sample to release polynucleotides present therein, analysing the sample using nucleic acid amplification to amplify the polynucleotides, and providing a result in 45 minutes or less, e.g., 40 minutes or less, 30 minutes or less, or 15 minutes or less identifying the presence, absence and/or abundance of each of the N microorganisms in the sample.

In embodiments, the method includes (i) determining microbiome data related to presence and or abundance of one or more microorganisms present in blood, tissue, plaque or other material obtained from the heart, (ii) determining information related to a condition of the heart based on the microbiome data; and (iii) determine an intervention, e.g., a therapeutic or mechanical (e.g., placing a stent within the subject) treatment in respect of the condition. The method can be performed during a catheterization or other cardiac intervention on a patient, accordingly, the intervention determined from the microbiome data can be acted upon during such cardiac intervention. That is, there is no need to re-open the patient.

In embodiments, the methods include developing underlying data: (i) in each of a number N cardiac interventions, e.g., catheterizations, obtain a sample, e.g., blood, tissue, plaque, or other material from a heart, (ii) in each of the number N samples, determine microbiome data related to presence and or abundance of one or more microorganisms; (iii) determine a relationship or other correlation between the microbiome data and a condition, e.g., a cardiac condition, of the patient.

In embodiments, the sample is obtained from one or more sites of the animal known or suspected to be associated with a cardiac condition, e.g., blood, tissue, plaque or other material obtained from the heart.

In embodiments of the invention, a method includes amplifying polynucleotides of at least N different microorganisms present in a sample obtained from an animal and determining, based on the amplified polynucleotides, microbiome data. The microbiome data includes data indicative of at least one of a presence and an abundance of each of the N microorganisms in the sample.

In embodiments of the invention, a device is configured to receive a sample including polynucleotides of at least N different microorganisms and to amplify the polynucleotides. The device includes a detector to detect at least one of a presence and an abundance of each of the polynucleotides. The device also includes a processor configured to determine microbiome data indicative of the presence and/or abundance of the microorganisms based on the detected polynucleotides.

In embodiments, animal is a human being. In other embodiments, the animal is an animal of agricultural importance, e.g., a fowl, bovine, or porcine, or an animal of human companionship, e.g., a feline or canine.

In embodiments, the processor is configured to determine the abundance of a microorganism as an absolute abundance, e.g., a number per volume, or a relative abundance, e.g., an abundance of a microorganism relative to an abundance of one or more other microorganisms in the sample or other parameters. As determined by the processor, data indicative of the presence of a microorganism may indicate the absence of such microorganism.

In embodiments, the device is configured to amplify the polynucleotides isothermally, e.g., without subjecting the polynucleotides to thermal cycling. For example, the device may include a heat source and temperature regulator configured to maintain a temperature of the polynucleotides to within +/−20° C., to within +/−15° C., to within +/−10° C., to within +/−5° C., to within +/−2.5° C., or at a substantially constant temperature during amplification.

In embodiments, the device is configured to amplify the polynucleotides without subjecting the polynucleotides to a temperature sufficient to denature double stranded polynucleotides during the amplification. For example, the temperature regulator of the device may be configured to prevent the temperature of the polynucleotides from exceeding about 90° C., about 80° C., about 70° C., or about 60° C. during amplification.

In embodiments, the device is configured to amplify the polynucleotides without first subjecting the polynucleotides to a temperature sufficient to denature double stranded polynucleotides present in the sample. For example, the device may be configured to receive the sample including the polynucleotides and to amplify the polynucleotides without first subjecting the polynucleotides to a temperature in excess of about 90° C., about 80° C., about 70° C., about 60° C., or about 55° C. In embodiments, the device is configured to detect the polynucleotides and/or amplicons without first subjecting the polynucleotides to such excess temperatures.

In embodiments, the device is configured to isothermal amplify the polynucleotides using recombinase polymerase amplification (RPA) or nicking and extension amplification reaction (NEAR).

In embodiments, the device is configured to amplify the polynucleotides in a total time T of about 45 minutes or less, about 30 minutes or less, about 20 minutes or less, or about 15 minutes or less.

In some aspects, the invention relates to the microbiome of a habitat of or a source of food or water for animals such as humans.

In embodiments of the invention, a method includes amplifying polynucleotides of at least N different microorganisms present in a sample obtained from the habitat or source of food or water and determining, based on the amplified polynucleotides, microbiome data. The microbiome data includes data indicative of at least one of a presence and an abundance of each of the N microorganisms. The method also includes determining, based on the microbiome data and on prior data related to the N microorganisms, data related to a condition of the habitat or source of food or water.

In embodiments, the habitat is an agricultural location, e.g., a location for the growth or production of agricultural products or livestock.

In embodiments, the prior data includes data indicative of a correlation between the presence or abundance of the N microorganisms and the condition of the habitat or source of food or water. For example, the data indicative of the condition of the habitat or source of food or water may include data indicative of how the presence and abundance of such microorganisms in the sample differ from a presence and abundance of the microorganisms found to be indicative of the suitability of the habitat for or source of food or water to support the growth or production of agricultural products or livestock.

In some aspects, the invention relates to determining data indicative of a correlation between a microbiome of an animal and a condition of the animal.

In embodiments of the invention, a method includes amplifying polynucleotides of at least N different microorganisms present in a sample obtained from each of a number N′ animals and determining, based on the amplified polynucleotides, microbiome data. The microbiome data includes data indicative of at least one of a presence and an abundance of each of the N microorganisms. The method includes determining condition data indicative of a condition of each of the N′ animals. The method also includes determining, based on the microbiome data and on the condition data, prior data related to the correlation between the microbiome data of an animal and a condition of the animal.

In embodiments of the invention, a method includes isothermally amplifying polynucleotides of at least N different microorganisms present in a sample obtained from the heart of a human subject, wherein N>1 and determining, based on the amplified polynucleotides, microbiome data comprising data indicative of at least one of a presence and an abundance of each of the N microorganisms. The method also includes determining, based on the microbiome data and on prior data related to the N microorganisms, data related to a condition of the animal.

In embodiments of the invention, a method includes performing isothermal nucleic acid amplification to detect N different microorganisms in a sample obtained from the heart obtained from a subject, determining microbiome data related to presence and or abundance of one or more microorganisms present in the sample, determining information related to a condition of the heart based on the microbiome data; and intervening in the subjects care with a therapeutic or mechanical treatment for the cardiac condition based on the microbiome data. In embodiments, the subject is a human subject.

In embodiments of the invention, a method includes determining information related to a cardiac condition of a patient, comprising (i) in each of a number N cardiac interventions, e.g., catheterizations, obtain a sample from the heart of the subject, (ii) in each of the number N samples, determine microbiome data related to presence and or abundance of one or more microorganisms, and (iii) determine a relationship or other correlation between the microbiome data and a cardiac condition of the subject.

In embodiments, the sample is obtained from one or more sites of the animal. The sample may include blood, tissue, plaque or other material obtained from the heart of the subject.

In embodiments, the cardiac condition is selected from the group consisting of atherosclerosis and atherosclerotic associated disease, e.g., an atherosclerosis-associated disease selected from the group consisting of coronary artery disease, myocardial infarction, angina pectoris, stroke, cerebral ischemia, intermittent claudication, gangrene, mesenteric ischemia, temporal arteritis, and renal artery stenosis. In embodiments, the cardiac condition is cardiovascular disease.

The method also includes determining a therapeutic or mechanical treatment for the cardiac condition based on the microbiome data and the prior data, e.g., the placement of a stent within an artery of the subject.

In embodiments, the method also includes recommending a treatment for the cardiac condition based on the microbiome data and the prior data.

In embodiments, the method is performed during a catheterization or other cardiac intervention on a subject.

In embodiments, the method also includes determining a therapeutic or mechanical treatment for the cardiac condition based on the microbiome data and the prior data, e.g., the placement of a stent within an artery of the subject.

In embodiments, the method also includes recommending a treatment for the cardiac condition based on the microbiome data and the prior data.

In embodiments, the method is performed during a catheterization or other cardiac intervention on a subject.

In embodiments, the method also includes determining a relationship or other correlation between the microbiome data and a cardiac condition further comprises considering prior data.

In embodiments, the method also includes recommending a treatment for the cardiac condition based on the microbiome data and the prior data.

### BRIEF DESCRIPTION OF THE FIGURES

FIG. 1 depicts a flow diagram representing a process to analyse a sample to generate a microbiome report.

FIG. 2 depicts a flow diagram representing a process to generate a microbiome report for an oral sample to identify a subject at potential risk of caries.

FIG. 3 depicts a flow diagram representing a process to generate a microbiome report for a scalp sample to identify a subject likely to be susceptible to scalp conditions, such as dandruff.

FIG. 4 depicts a flow diagram representing a process to identify microbiome data associated with acute clinical indications.

FIG. 5 depicts an assay device.

### DETAILED DESCRIPTION

Methods and devices are disclosed for determining microbiome data indicative of the presence or abundance of microorganisms present in a microbiome of a person. The microbiome data are used in conjunction with prior data to determine data related to a health condition of the person. The prior data include data indicative of a correlation between the presence or abundance of the microorganisms in the microbiome of humans and the presence, absence or severity of or a tendency toward the condition. Thus, for example, the data related to the health condition of the person may indicate that the person has a presence or abundance of microorganisms similar to persons having a tendency to develop a particular health condition. Alternatively, the data may indicate that the person has a presence or abundance of microorganisms similar to persons who are suitable for a particular treatment or therapeutic regimen for the health condition. Based on these data, action is taken with respect to the person and the health condition. For example, the person make be given a therapeutic regimen intended to reduce a probability that the person will develop the particular health condition that the person has a tendency towards. Alternatively, the person may be given the treatment or therapeutic regimen identified as suitable for the health condition. Advantageously, the methods and devices described herein permit microbiome data of a person to be determined quickly, e.g., in 45 minutes or less, at the point of care, e.g., at a location remote from a central laboratory, and using instruments that can be operated by individuals without substantial training.

Polynucleotides suitable for amplification in connection with the present methods and devices include double-stranded and single-stranded nucleic acid molecules, such as DNA and RNA molecules. The polynucleotides may be of genomic, chromosomal, plasmid, mitochondrial, cellular, and viral nucleic acid origin. For double stranded polynucleotides, the amplification may be of either one or both strands.

Isothermal polynucleotide amplification technologies suitable for use in the present methods and devices include nicking and extension amplification reaction amplification as described in U.S. application Ser. No. 11/778,018 filed 14 Jul. 2007 (the “'018 Application”, Attached hereto as Appendix A) and recombinase polymerase amplification as described in U.S. Pat. No. 7,399,590 (the “'590 Patent”, Attached hereto as Appendix B). Isothermal technologies for performing multiplexed amplification of polynucleotides include multiplexed recombinase polymerase amplification as described in U.S. Pat. No. 8,580,507 (the “'507 Patent”, attached hereto as Appendix C) and multiplexed recombinase amplification as described in an article by Ming et al. in ACS Nano with a publication date of February 2015, attached hereto as Appendix D). Each of the foregoing references is incorporated herein by reference in its entirety and considered part of the present disclosure.

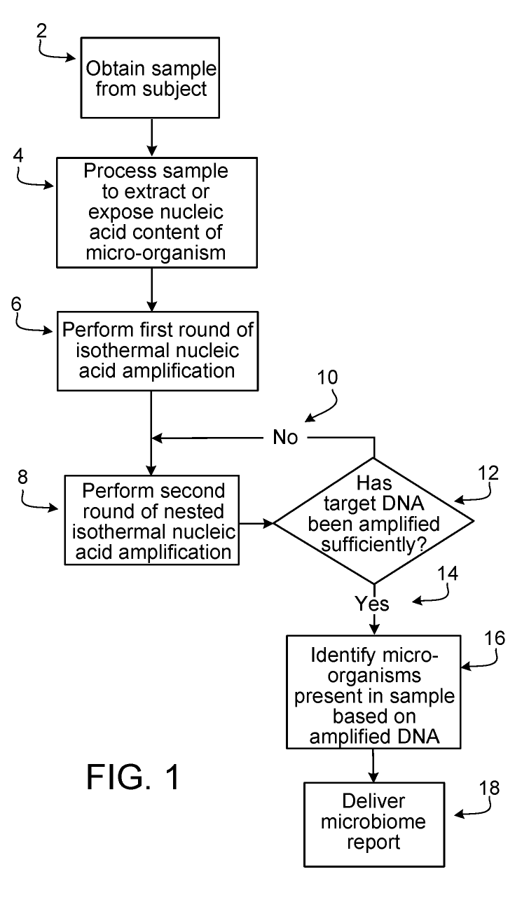

With reference to FIG. 1, a method for preparing a microbiome report including microbiome data includes a step 2 of obtaining a sample from a subject, a step 4 of processing the sample to extract or expose nucleic acid content of one or more microorganisms, performing a first round of isothermal nucleic acid amplification 6, a step 12 of determining whether target nucleic acids (e.g., DNA) of the microorganisms have been sufficiently amplified, if not 10, a second round of isothermal nucleic acid amplification 8 is performed, which may be nested. If the determination in step 12 is that sufficient amplification has been performed, amplified nucleic acid is detected 16 and the microorganisms present in the sample identified. A microbiome report 18 containing microbiome data indicative of the presence, absence or abundance of microorganisms is generated.

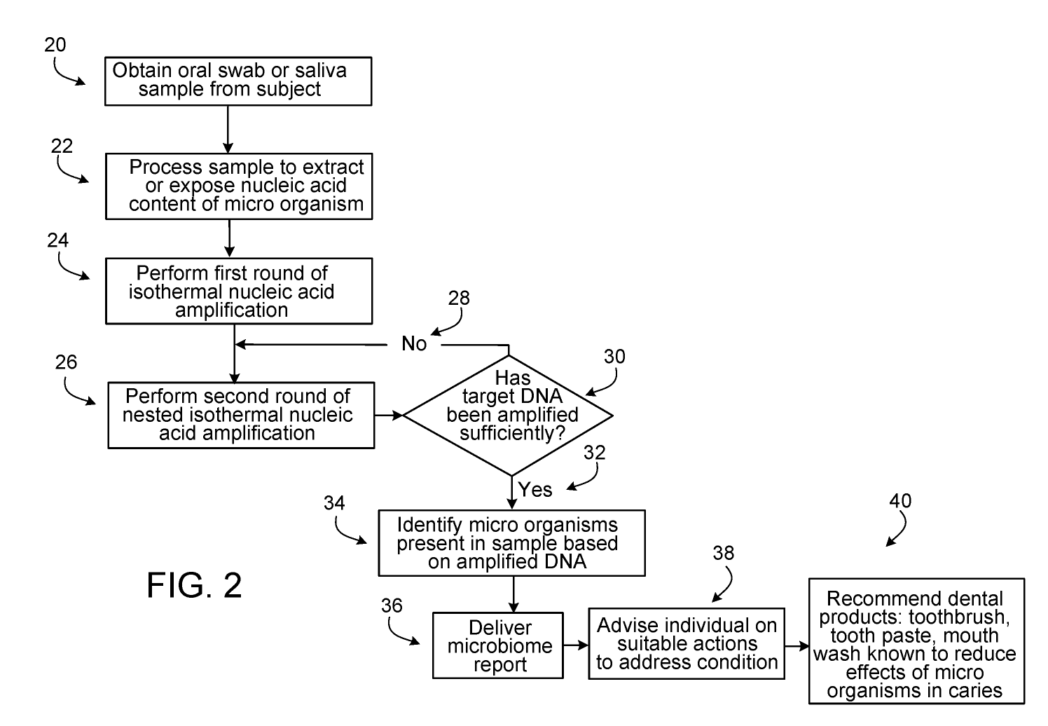

With reference to FIG. 2, a method is described for depicts a method for generating a microbiome report including microbiome data indicative of a condition of an oral cavity of an animal, e.g., a human. The method includes a step 20 of obtaining a sample from an oral cavity a subject. Typical samples include saliva and swabs of tissue, as from a cheek or gum. A step 22 of processing the sample is performed to extract or expose nucleic acid content of one or more microorganisms, performing a first round of isothermal nucleic acid amplification 24, a step 30 of determining whether target nucleic acids (e.g., DNA) of the microorganisms have been sufficiently amplified, if not 28, a second round of isothermal nucleic acid amplification 26 is performed, which may be nested. If the determination in step 28 is that sufficient amplification has been performed, amplified nucleic acid is detected 34 and the microorganisms present in the sample identified. A microbiome report 36 containing microbiome data indicative of the presence, absence or abundance of microorganisms related to a condition of the oral cavity of the subject is generated. In a step 28, the individual is advised as to the condition of the oral cavity and dental products recommended 40.

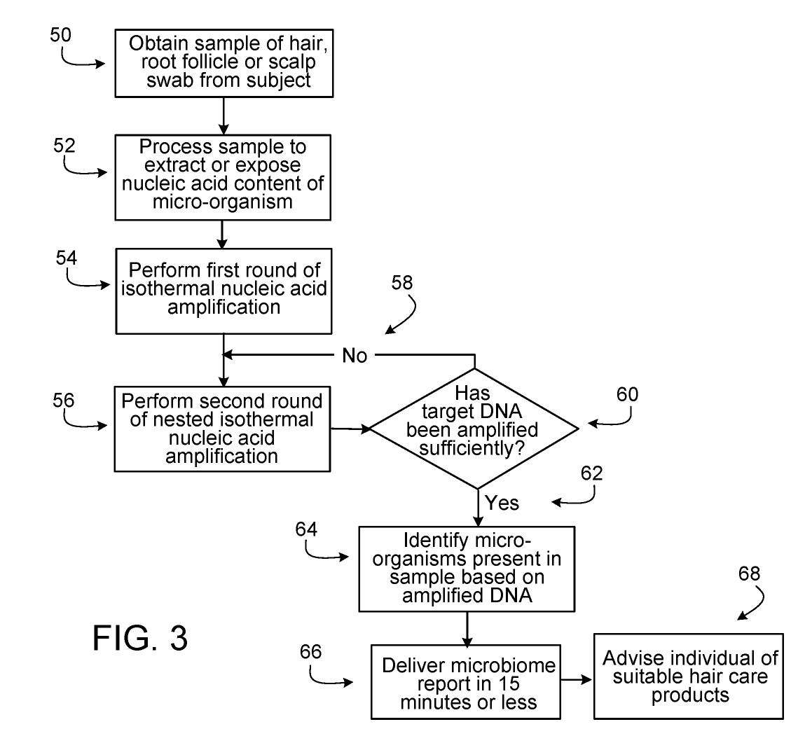

With reference to FIG. 3, a method is described for depicts a method for generating a microbiome report including microbiome data indicative of a condition of a scalp or hair of an animal, e.g., a human. The method includes a step 50 of obtaining a sample from an oral cavity a subject. Typical samples include hair, root follicle, skin, or a scalp swab. A step 52 of processing the sample is performed to extract or expose nucleic acid content of one or more microorganisms, performing a first round of isothermal nucleic acid amplification 54, a step 60 of determining whether target nucleic acids (e.g., DNA) of the microorganisms have been sufficiently amplified, if not 58, a second round of isothermal nucleic acid amplification 56 is performed, which may be nested. If the determination in step 58 is that sufficient amplification has been performed, amplified nucleic acid is detected 64 and the microorganisms present in the sample identified. A microbiome report 66 containing microbiome data indicative of the presence, absence or abundance of microorganisms related to a condition of the scalp, hair, or skin of the subject is generated. In a step 68, the individual is advised as to the condition of the scalp, hair, or skin and suitable medicaments or care cosmetic products recommended.

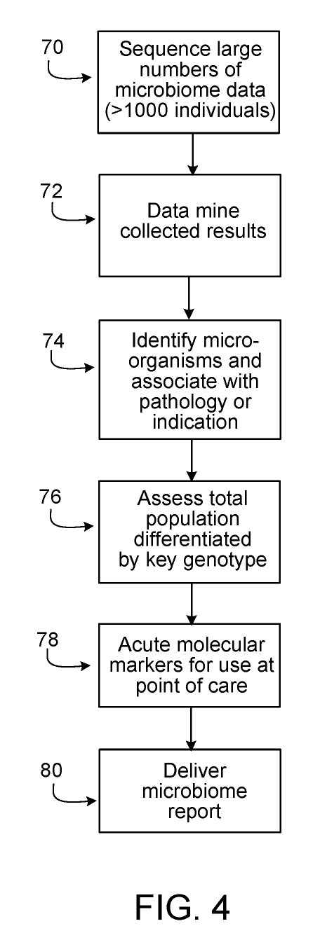

With reference to FIG. 4, a method is described for identifying microbiome data associated with a condition of animals, e.g., acute clinical indications. In a step 70, microbiome data is obtained from each of a number of animals. The microbiome data may include any or all of data indicative of the presence, absence, abundance, or sequence of polynucleotides of each of a number N microorganisms. In a step, 72, the microbiome data and data indicative of a condition of each of the animals is analysed to determine a correlation between the microbiome data and the condition. For example, the data indicative of the condition may include determining 74 data indicative of a correlation between the presence, absence or abundance of the number N microorganisms or a subset thereof and the status of the condition. The method may include determining 76 a genotype of each of the N microorganisms or a subset thereof. The method includes determining 78 molecular markers useful in determining a prognosis, treatment, or status of a condition of an animal.

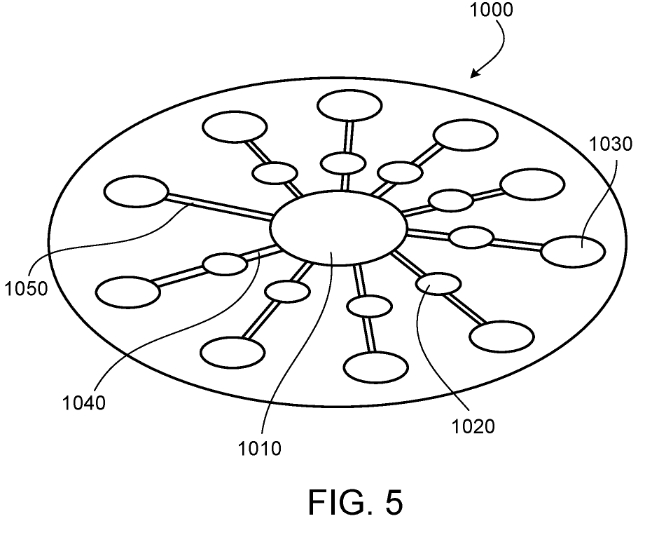

FIG. 5 shows an assay disc 1000 which may be used to perform analyses of a plurality of samples to determine a microbiome profile for each sample. Assay disc 1000 comprises a central reaction chamber 1010, one or more second chambers 1020, and one or more third chambers 1030, each connected by fluidic channels 1040, 1050. Central reaction chamber 1010 may contain one or more reagents, which may be liquid reagents or dried reagents, necessary to perform a first isothermal nucleic acid amplification. The one or more second chamber(s) 1020 are connected to the central reaction chamber by fluidic channel 1040, and the one or more third chamber(s) 1030 are connected to the one or more second chamber(s) 1020 by fluidic channel 1050. Second chamber 1020 may comprise liquid or dried reagents for performing a specific isothermal nucleic acid amplification, in which a unique primer may be present to cause amplification of DNA known to be unique to a specific microbial species. Third chamber 1030 may further comprise measurement electrodes or may be an optical cell in which measurement or determination of the quantity or presence of amplified DNA indicative of a specific microorganism is done.

In use, a sample is applied to the central reaction chamber 1010 of disc 1000, which is rotated at a first rotational velocity. Sample is mixed with reagent within central reaction chamber 1010 in order to lyse the sample to release genomic DNA and thereafter initiate a first round of isothermal nucleic acid amplification. During the first round of isothermal nucleic acid amplification a bulk amplification of all nucleic acid material released from microbial species present in the sample occurs. Following the first round of amplification the disc 1000 may be rotated at a greater velocity, sufficient to cause the liquid sample to transfer through fluidic channel 1040 into second chamber 1020. As a result of the arrival of sample in second chamber 1020, a second round of isothermal nucleic acid amplification is initiated. In this instance amplification of a specific DNA sequence occurs through use of specific primers for a particular microorganism. For example, one chamber might contain primers for E. coli, another chamber may contain primers for methicillin resistant S. aureus (MRSA), another chamber may contain primers for C. albicans, etc. Once the second round of amplification has completed, the disc 1000 may be rotated at a yet higher velocity thus causing liquid sample to be transferred via fluidic channel 1050 into third chamber 1030, within which the presence of the specifically amplified DNA is determined. In one embodiment determination may be performed optically, using fluorescence, luminescence, UV or visible spectrometry, in either reflectance or transmission mode. In another embodiment determination may be performed using electrochemistry.

### EXAMPLES

### 1. Dental Care

A patient visits a dental office. A dental technician obtains a saliva sample from the patient and proceeds to clean the patient's teeth. Meanwhile, a technician introduces the sample to a cartridge of a point of care polynucleotide amplification instrument located in the dental office. The cartridge includes reagents configured to amplify and detect the following microorganisms: Firmicutes, Bacteriodetes, Proteobacteria, Actinobacteria, Fusobacteria, Streptococcus, Abitrophia, Gemella, Granulicatella, Prophyromonas and P. gingivalis. A first set of the reagents are present in lyophilized form within the cartridge and are mobilized upon introduction of the sample. The first set of reagents including lysing agents configured to lyse cells present in the sample and primers and probes configured to amplify polynucleotides associated with the microorganisms. A second set of reagents include a second set of primers and probes immobilized with respect to an array defined on an inner surface of a chamber within the cartridge. The second set of reagents include primers configured to amplify amplicons produced by reaction of the first set of reagents and the polynucleotides of the microorganisms.

The cartridge also includes a liquid buffer configured to combine with and dilute the sample. Upon the introduction to the cartridge, the sample combines with the liquid buffer and the resulting mixture dissolves and mobilizes the first set of reagents. The resulting mixture is introduced to the chamber containing the immobilized reagents. The chamber is heated to a maximum temperature of less than 65° C. and, once heated, the temperature is maintained at a substantially constant temperature. Reaction of the polynucleotides of the microorganisms and the first set of reagents produces a first set of amplicons which then begin to react with the primers of the second, immobilized set of reagents. This reaction produces a second set of amplicons substantially immobilized with respect to sits of the array. Second reagents associated with each site of the array are configured to amplify and permit detection of polynucleotides associated with a different one of the microorganisms. After less than 45 minutes following the introduction of the sample to the cartridge, a light source of the point of care instrument illuminates the array and a detector measures fluorescence emitted from the respective sites thereof. A processor within the instrument determines fluorescence data based on the measured fluorescence.

The processor of the instrument determines microbiome data indicative of the presence and an abundance of each of the microorganisms and compares the microbiome data to prior data indicative of a correlation between the presence and/or abundance of the microorganisms and a tendency of a human to develop periodontal disease. Data indicative of the patient's tendency to develop periodontal disease are transmitted to a dental record associated with the patient.

As the dental technician finishes cleaning the patient's teeth, a dentist caring for the patient examines the patient's mouth reads the comparison data and then informs the patient that treatment with an antibacterial medicament configured to alter a relative abundance of the microorganisms of the patient's mouth is necessary to reduce the likelihood of the patient developing periodontal disease. The medicament is administered to the patient at the end of the patient's visit to the dental office.

### 2. Dermatology

A dermatologist examines a patient's acne and collects a sample swab from an affected area of skin. While the dermatologist continues the examination, a technician introduces sample from the swab to a cartridge of a point of care polynucleotide amplification instrument located in the dermatologist's office. The instrument includes features corresponding to the instrument described in Example 1, except that the cartridge includes reagents configured to amplify and detect propionibacterium acnes and other microorganisms associated with acne and other skin conditions. Within 45 minutes, the instrument isothermally amplifies the polynucleotides associated with the microorganisms and determines microbiome data indicative of the presence and an abundance of each of the microorganisms and compares the microbiome data to prior data indicative of a correlation between the presence and/or abundance of the microorganisms and a tendency of a human toward skin diseases include acne. Data indicative of the patient's tendency to develop periodontal disease are transmitted to a medical record associated with the patient.

Before the patient has left the dermatologist office, the dermatologist reads the comparison data and then informs the patient that treatment with a medicament configured to alter a relative abundance of the microorganisms present in the areas of the patient's skin affected by acne will reduce the severity of the disease. The dermatologist prescribes the medicament to the patient at the end of the patient's visit.

### 3. Cosmetic Selection

A customer visits a store providing personal care products including cosmetics. The customer informs an employee that the customer has recently begun to experience dry skin. The employee learns that the customer has been using an antibacterial body wash. The employee takes a swab from a portion of the affected skin of the customer and introduces sample from the swab to a cartridge of a point of care polynucleotide amplification instrument located in the store. The instrument includes features corresponding to the instrument described in Example 1, except that the cartridge includes reagents configured to amplify and detect Propionibacterium acnes, Actinobacteria, Firmicutes, Proteobacteria, Bacteroidetes and other microorganisms associated with skin conditions. Within 45 minutes, the instrument isothermally amplifies the polynucleotides associated with the microorganisms and determines microbiome data indicative of the presence and an abundance of each of the microorganisms and compares the microbiome data to prior data indicative of a correlation between the presence and/or abundance of the microorganisms and a tendency of a human toward skin conditions including dry skin and suitability for particular skin care products. The data are displayed to the employee and printed or electronically transmitted (e.g., to an email address of the customer) for retention. The employee may then inform the customer of various preparations and medicaments suitable for ameliorating the dry skin condition.

### 4. Pre-Diabetes/Diabetes Management

An individual visits a healthcare provider facility and provides a sample of blood and a sample of faeces. The healthcare provider analyses the sample of blood to determine a blood glucose measurement and an HbA1c measurement. The healthcare provider also analyses the faecal sample using an instrument that includes features corresponding to the instrument described in Example 1, with additional sample processing means to separate solids content to yield a clarified solution in which the microbial flora is suspended. The assay cartridge includes reagents configured to amplify and detect a range of enteric microbial species, including Firmicutes, Bacteroidetes, Actinobacteria, and Proteobacteria, and other microorganisms associated with the digestive tract. Within 45 minutes, the instrument isothermally amplifies the polynucleotides associated with the microorganisms and determines microbiome data indicative of the presence and an abundance of each of the microorganisms and compares the microbiome data to prior data indicative of a correlation between the presence and/or abundance of the microorganisms and a provides a dietary recommendation for the individual which would be expected to result in a reduction in post-prandial hyperglycaemia as well as resulting in a generally lowered HbA1c over time.

Studies have shown there to be direct links between dietary composition and post-prandial blood glucose as a function of microbiome composition. Studies have also indicated that consumption of certain food supplements, such as for example sugar alternatives including saccharin, may in fact result in an adversely elevated blood glucose level as a result in an altered microbiome state induced by a reduced level of sugar and increased level of artificial sweetener in the diet.

Based on the microbiome data the healthcare provider may subsequently provide the individual with recommended modifications to their diet which are intended to promote certain microbial species and inhibit other species that are known to result in elevated blood glucose due to the way in which such species metabolise complex carbohydrates, releasing greater quantities of glucose that might subsequently be adsorbed through the intestinal wall into the blood stream.

Frequent monitoring of blood glucose, HbA1c and microbiome composition by the healthcare provider may lead to a long term reduction in HbA1c, and in some cases prevention of the development of type 2 diabetes or amelioration of type 2 diabetes.

1. A method, comprising:
a) amplifying polynucleotides of at least N different microorganisms present in a sample obtained from an animal, wherein N>1;
b) determining, based on the amplified polynucleotides, microbiome data comprising data indicative of at least one of a presence and an abundance of each of the N microorganisms; and
c) determining, based on the microbiome data and on prior data related to the N microorganisms, data related to a condition of the animal.

2. The method of claim 1, wherein the animal is a human.

3. The method of claim 1, further comprising determining a treatment for the condition based on the microbiome data and the prior data.

4. The method of claim 3, further comprising administering the treatment to the animal.

5. The method of claim 1, wherein the amplification is performed isothermally.

6. The method of claim 5, wherein the amplification is performed without subjecting the polynucleotides to a temperature in excess of about 75° C.

7. The method of claim 1, wherein the amplification is performed without first subjecting the polynucleotides to a temperature sufficient to denature double stranded polynucleotides present in the sample.

8. The method of claim 1, wherein the amplification is performed using recombinase polymerase amplification or nicking and extension amplification reaction.

9. The method of claim 1, wherein the amplification is performed in a total time T of about 30 minutes or less.

10. The method of claim 1, wherein N is at least 5.

11. The method of claim 1, wherein the prior data are indicative of a correlation between the presence or abundance of the microorganisms in humans and the presence, absence or severity of or a tendency toward the condition in such humans.

12.-15. (canceled)

16. A method of screening a sample obtained from a human subject at point of care or point of presence to determine the identity of one or more microorganisms present or potentially present in the sample, the method comprising the steps;
obtaining a sample from the human subject; processing the sample;
applying the processed sample to an assay device;
operating the assay device to perform at least one isothermal nucleic acid amplification cycle to amplify the nucleic acid content of the sample;
performing further isothermal nucleic acid amplification cycles to amplify defined sequences if present know to be associated with particular microorganisms;
identifying the microorganisms present in the sample based on the amplified nucleic acid; and
reporting to a user the specie or species of microorganism present in the sample.

17. The method of claim 16, wherein the step of processing the sample comprises lysing the sample to release nucleic acid contents; filtering the sample to remove debris which may interfere with nucleic acid amplification; and optionally concentrating the sample.

18. The method of claim 16, wherein the step of applying the processed sample to an assay device comprises delivering the processed sample to a reaction chamber to initiate isothermal nucleic acid amplification.

19. The method of claim 16, wherein the process of isothermal nucleic acid amplification comprises recombinase polymerase amplification (RPA), nicking and extension amplification reaction (NEAR).

20. The method of claim 16, wherein screening of the sample is performed to identify one or more microorganisms known to be present on the skin of a human being.

21. The method of claim 16, wherein screening of the sample is performed to identify one or more microorganisms known to be present on or around the eye of a human being.

22. The method of claim 16, wherein screening of the sample is performed to identify one or more microorganisms known to be present in the upper respiratory tract of a human being.

23. The method of claim 16, wherein screening of the sample is performed to identify one or more microorganisms known to be present in the intestinal tract of a human being.

24. The method of claim 16, wherein screening of the sample is performed to identify one or more microorganisms known to be present in the oral cavity of a human being.

25. The method of claim 16, wherein screening of the sample is performed to identify one or more microorganisms known to be present in the genitourinary tract of a human being.

26. The method of claim 1, wherein based on the microorganisms identified offering to said subject a range of one or more products that may benefit the wellbeing of the subject.

27. The method of claim 26, wherein the offering of products comprises offering cosmetic products for application to the facial area, offering anti-perspirant products, offering deodorant products.

28. The method of claim 27, wherein the offering of products comprises offering a probiotic, offering a pre-biotic, offering an antibiotic.

29. A method of determining the microbiome profile of an individual comprising receiving a sample from the individual, performing isothermal nucleic acid amplification of a nucleic acid associated with at least one microorganism, and detecting the amplified nucleic acid product, wherein the detected products are used to build a profile of the individual's microbiome.

30. The method of claim 29, wherein the step of determining the microbiome profile is completed in 15 minutes or less, 10 minutes or less, five minutes or less, 3 minutes or less.

31. The method of claim 29, further comprising providing a set of algorithms for linking the data associated with the individual's microbiome to a certain medical condition, physical condition, or likely responsiveness to a certain therapy.

32. The method of claim 16, wherein from the output of the method, selecting a compound that is synergistic with the microbial species or a compound produced by the microbial species.

33. The method of claim 16, wherein based on the result of the method, selecting a compound to treat the medical condition, physical condition, or according to individual's responsiveness to the therapeutic use of the compound.

34. The method of claim 16, wherein the data associated with an individual's microbiome comprises data about the environment.

35. The method of claim 34, wherein the selected compound is compatible with the environment.

36. The method of claim 16, wherein the isothermal amplification of DNA is performed using a cassette.

37. The method of claim 16, wherein the isothermal amplification is multiplexed amplification of a single sample.

38. The method of claim 16, wherein a single sample is divided into a plurality of sample fractions, wherein each fraction is combined with reagents for isothermal amplification and primers specific to at least one microorganism.

39. The method of claim 16, wherein the step of detecting comprises detecting a 10⁶ fold amplification, a 10⁷ fold amplification, a 10⁸ fold amplification, a 10⁹ fold amplification, a 10¹⁰ fold amplification, a 10¹¹ fold amplification, a 10¹² fold amplification of the target nucleic acid sequence.

40. The method of claim 16, wherein the method is performed at point of care, or point of retail.

41. The method of claim 16, wherein the sample is selected from the group comprising; blood, plasma, serum, urine, saliva, tear fluid, seminal fluid, a vaginal swab, a nasal swab, a rectal swab, a skin swab, an in ear swab, a cheek swab, a throat swab, a fecal sample, cerebrospinal fluid.

42. The method of claim 16, wherein the method comprises determining the susceptibility of an individual to a therapy based on synergism of compound with the microbial species identified on said individual.

43. The method of claim 16, wherein the compound used to treat the medical or physical condition affects the count of a bacterium found in the individual's microbiome profile.

44. The method of claim 43 wherein the compound used to treat comprises a pre-biotic, a pro-biotic or an antibiotic.

45.-111. (canceled)
